(19) United States
(12) Patent Application Publication
(10) Pub. No.: US 2007/0275005 A1
(43) Pub. Date: Nov. 29, 2007

(54) Use of Lipopeptides for Activating T Lymphocytes Through the Skin

(75) Inventors: Herve Groux, Biot (FR); Valerie Brun, Biot (FR); Arnaud Foussat, Biot (FR)

Correspondence Address: FOLEY AND LARDNER LLP;SUITE 500, 3000 K STREET NW, WASHINGTON, DC, 20007, US

(73) Assignees: TX-CELL; INSTITUT NATIONAL DE LA SANTE ET DE LA RECHERCHE MEDICALE (INSERM)

(21) Appl. No.: 10/579,078

(22) PCT Filed: Nov. 5, 2004
(86) PCT No.: PCT/IB04/03882

§ 371 Date: Jun. 16, 2007

(30) Foreign Application Priority Data
Nov. 12, 2003 (FR) 0313261

(51) Int. Cl. A61K 39/395; A61P 37/08; C07K 16/28

(52) U.S. Cl. 424/193.1; 514/2; 424/144.1

(57) ABSTRACT

The present invention relates to the use of a method of treating or preventing a disease selected from the group of skin and diseases of the mucosa, comprising administering topically to a mammal in need of such a treatment a topical medicinal product comprising a lipopeptide or a mixture thereof, wherein said lipopeptide comprises a peptide antigen specific for a T cell population, said peptide antigen being coupled covalently with a lipid radical and being capable of activating the T cell population. Such a use is more specifically intended for a transcutaneous application of the topical medicinal product, which is advantageously intended to prevent or treat a skin disease. The invention also relates to pharmaceutical or cosmetic formulations comprising the lipopeptide according to the invention.

The present invention relates to the use of a method of treating or preventing a disease selected from the group of skin diseases and diseases of the mucosa, comprising administering topically to a mammal in need of such a treatment a topical medicinal product comprising a lipopeptide or a mixture thereof, wherein said lipopeptide comprises a peptide antigen specific for a T cell population, said peptide antigen being coupled covalently with a lipid radical and being capable of activating the T cell population. Such a use is more specifically intended for a transcutaneous application of the topical medicinal product, which is advantageously intended to prevent or treat a skin disease. The invention also relates to pharmaceutical or cosmetic formulations comprising the lipopeptide according to the invention.

It is desired to be able to find a way to induce an immune response through the skin, in particular, it is desired to activate T lymphocytes, either with a view to activating defence mechanisms, or with a view to regulating a mediated T immune response.

The need to induce such an immune response is particularly justified in skin cancers such as melanoma or to boost immunisation.

In parallel, it may be necessary to regulate the immune response for numerous skin diseases, such as psoriasis, vitiligo, prurigo, pityriasis, eruptive cutaneous mastocytosis, scleroderma, bullate dermatosis, cutaneous emphysema, eczema or acne, or for inflammatory skin reactions, such as for example in cases of oedema, graft rejections, or following a burn, radiation, a cut, sting or due to an allergen or microbe.

Topical applications of proteins or peptides capable of inducing transcutaneous immunisations are currently known, but these methods are, firstly, not effective and, secondly the peptides used may represent risks for human health (Glenn et al, Nature, Vol. 391, 26 February 1998, p.851; Glenn et al, Nat Med. 2000 December; 6(12):1403-6; Hammond et al, Vaccine. Mar. 21, 2000; 19(17-19):2701-7).

Independently, lipopeptides, consisting of a peptide compound bound covalently to a non-peptide lipophilic part, are well-known to those skilled in the art. They were initially developed to solve the problem of the entry of substances comprising pharmacological properties. In fact, synthetic peptides and oligonucleotides have difficulty in passing the cell membrane. A beneficial approach to improving their ability to penetrate the cell is to modify them with a lipophilic part.

The inventors very surprisingly discovered that the topical administration of lipopeptides comprising an antigenic peptide on the skin was capable of activating T lymphocytes locally, very effectively and without any risks for health.

In this way, according to a first aspect, the present invention relates to a method of treating or preventing a disease selected from the group of skin diseases and diseases of the mucosa, comprising administering topically to a mammal in need of such a treatment a topical medicinal product comprising a lipopeptide or a mixture thereof, wherein said lipopeptide comprises a peptide antigen specific for a T cell population, said peptide antigen being coupled covalently with a lipid radical and being capable of activating the T cell population.

According to the present invention, the peptide antigen of the lipopeptide is a non-pathogenic immunogenic peptide that may be either a MHC class I restricted peptide to prime a CD8+ T lymphocyte mediated immune response, or a MHC class II restricted peptide capable of enhancing an humoral response elicited by CD4+ T lymphocytes. It has to be noted that CD4+ T lymphocytes comprise Tr1 lymphocytes. For example, such non-pathogenic immunogenic peptides may be non-allergic food antigens or non-pathogenics bacterial antigens. MHC class I restricted peptides may be, for example, restricted to the H2-K^{b} molecule; MHC class II restricted peptides may be, for example, restricted to the I-A^{d} molecule. The man skilled in the art will know which peptides may be used to enhance a CD8+ or a CD4+ T lymphocyte response.

There are several ways to use the lipopeptide of the present invention. In a first way, the lipopeptide is administered topically and in a repeated manner to activate a T cell population. In a second way, the peptide antigen or a polypeptide comprising the peptide antigen is administered prior the topical administration of the lipopeptide comprising the same peptide antigen, in a prior immunisation step.

Preferably, the administration of the peptide antigen for immunisation is made subcutaneously or intraperitoneally.

In a third way, a T cell population (also named herein a lymphocyte population) which has been previously in vitro activated by the peptide antigen is administered together with the topical administration of the lipopeptide comprising the same peptide antigen. The peptide antigen-actived T cell population and the lipopeptide may be administered sequencially, simultaneously or separately.

Preferentially, the peptide type antigen contains at least 6, preferentially at least 7, 8, 9, 10, 12, 13, 14, 15, 16, 17, 18, 19, 20, 25, 50, 100, 150, 200, 250, 300, 350 or at least 400 amino acids.

The terms “peptide”, “protein”, “polypeptide” and expressions such as “peptide antigen” are used indifferently in the present application to refer to a sequence of several amino acids.

The covalent coupling between the lipid radical and the peptide antigen may be carried out according to different methods known to those skilled in the art. For example, it is possible to mention, without being restrictive, coupling between a fatty acid and a solid phase peptide, as particularly described by K. THIAM et al. in Biochemical and Biophysical Research Communications, 1998, 253,639-647, coupling in solution of a protein with a palmotoyl-coenzyme A group, the latter being introduced into a cysteine thiol group, chemical ligation, which is used to bind, in solution and under extremely mild conditions, two previously purified and completely deprotected peptide structures, such as the disulphide bond for example.

W. ZENG et al. (J. Pept. Sc., 1996,2,66-72) also propose to bind, in solution, a completely deprotected and previously purified peptide with a polyfunctional lipid structure bound with a peptide, via an oxime bond. The lipophilic part is introduced onto a solid phase peptide sequence.

Similarly, O. MELNYK et al. (J. Peptide Res., 1998, 52, 180-184) described the ligation, in solution and with a hydrazone bond, between a peptide comprising a lipophilic chain and an aldehyde function and another peptide modified on the lateral lysine chain with a hydrazino group. The hydrazone bond is produced in solution and the peptide type lipophilic compound is synthesised in solid phase.

In addition, C. KLINGUER et al. (Tetrahedron Letters, 1996, 37, n 40, 7259-7262) described the ligation, in a water/acetonitrile mixture and with a hydrazone bond, between a peptide comprising a hydrazine function and cyclohexanecarboxaldehyde.

The covalent coupling may also consist of the creation of a hydrazide bond between the peptide and the compound coupled thereto, in convergent synthesis in solution. The method used is described in the international patent application published under the number WO 01/14408 and in D. BONNET et al. (Tetrahedron Letters, 2000, 41 45-48).

The coupling between the peptide and the lipid compound, produced in solution, may also consist of the creation of a hydrazide bond, as described in the international patent application published under the number WO 02/20558.

In a specific embodiment of the invention, the lipid radical is derived from a fatty acid. Examples of fatty acids include, without being restrictive, palmitic acid, stearic acid, oleic acid or linoleic acid. More preferentially, the fatty acid is palmitic acid.

To activate the T lymphocytes, it is possible to administer either a single type of lipopeptide according to the invention, or several types, said types varying according to the lipid radical and peptide antigen coupled thereto.

The activated T lymphocytes according to the present invention may be of various types, such as for example CD8+ T lymphocytes, CD4+ T lymphocytes, also referred to as T helper lymphocytes, or Tr1 regulator lymphocytes, which appear to be in a specific category of CD4+ T lymphocytes (Chen et al, 1994, Science 265, 1237-1240; Groux et al., 1997, Nature 389, 737-742; Mc Guirck et al, 2002, J Exp Med 195, 221-231; Powrie et al, 1.994, J Exp Med 179, 589-600).

Preferably, the T cell population is a CD8+ T cell population, a CD4+ T cell population or a Tr1 cell population.

In this way, for example, if the peptide antigen of the lipopeptide according to the invention administered is specific for Tr1 lymphocytes, the latter will be capable of recognising it and will be activated. They will then be able to exert their anti-inflammatory action.

In a specific embodiment of the invention, between 1 μg/cm² to 500 μg/cm², preferably 10 μg/cm², of said lipopeptide is administered topically to said mammal.

The lipopeptide may be administered daily during one to fourteen days, preferably during seven days.

In another specific embodiment of the invention, the topical medicinal product further comprises a pharmaceutically topical acceptable carrier.

The expression “pharmaceutically topical acceptable carrier”, as used herein, means that the carrier is suitable for topical application to the skin, has good aesthetic properties, is compatible with the lipopeptide of the present invention and any other components, and will not cause any untoward safety or toxicity concerns. A safe and effective amount of carrier is from about 40% to about 90%, preferably from about 45% to about 85%, more preferably from about 50% to about 80% of the topical medicinal product.

The carrier can be in a wide variety of forms. For example, emulsion carriers, including, but not limited to, oil-in-water (e.g. emulgel), water-in-oil, water-in-oil-in-water, and oil-in-water-in-siiicone emulsions, are useful herein. Preferred cosmetically and/or pharmaceutically acceptable topical carriers include oil-in-water emulsions.

These emulsions can also be delivered in the form of sprays using either mechanical pump containers or pressurized aerosol containers using conventional propellants. These carriers can also be delivered in the form of a foam. Other suitable topical carriers include anhydrous liquid solvents such as oils, and silicones; aqueous-based single phase liquid solvents; and thickened versions of these anhydrous and aqueous-based single phase solvents.

The topical medicinal product of the present invention is generally prepared by conventional methods such as are known in the art of making topical compositions.

Such methods typically involve mixing of the ingredients in one or more steps to a relatively uniform state, with or without heating, cooling, application of vacuum, and the like. Non-limiting examples of the product form can be a cream, paste, gel, emulsion, lotion, ointment, solution, liquid, etc.

In a most preferred embodiment, the topical medicinal product is intended to be administered at the inflammation site.

In another embodiment, the topical medicinal product may be suitable for epicutaneous application, for transmucosal application or for transcutaneous application.

The topical medicinal product may also additionally comprise one or more immunity adjuvants.

In a preferred embodiment, the method as depicted above further comprises, prior to the topical administration of the lipopeptide, an immunisation step of the mammal with the peptide antigen or with a polypeptide comprising the peptide antigen. Preferably, the prior immunisation is made by any appropriate route, preferably subcutaneously or intraperitoneally.

Another specific embodiment is the method of the invention wherein the peptide antigen has been used to activate in vitro, as a T cell population, a Tr1 cell population obtained from a CD4+ T cell population of said mammal, and wherein said method further comprises the administration of the Tr1 cell population activated by said peptide antigen, the topical administration of the lipopeptide being made sequencially, simultaneously or separately with the administration of the Tr1 cell population.

Preferably, the peptide antigen-activated Tr1 cell population which is administered to the mammal is from 10⁶ to 10⁹ cells/kg.

More preferably, the peptide antigen-activated Tr1 cell population which is administered to the mammal is from 0.5 10⁷ to 1.5 10⁷ cells/kg, preferably 10⁷ cells/kg.

The number and the frequency of the Tr1 cell administration will depend on the application. The man skilled in the art will know how to dose this cell population in an appropriate manner. The Tr1 cell population which is administered may be in any appropriate medium, preferably in a saline medium.

Another specific embodiment is the method of the invention comprising intravenous, intramuscular, intra-arterial, intramedullar, intrathecal, intraventricular, transdermal or subcutaneous administration of the peptide antigen-activated Tr1 cell population.

In the present invention, diseases selected from the group of skin diseases and diseases of the mucosa may comprise allergic, inflammatory and/or immune disorders, as well as auto-immune or chronic inflammatory diseases.

In a preferred embodiment, the skin disease is selected from the group comprising psoriasis, vitiligo, prurigo, pityriasis, eruptive cutaneous mastocytosis, scleroderma, bullous dermatitis, cutaneous emphysema, eryhtema, eczema, acne, oedema, graft rejection and melanoma

More preferably, the skin disease is a local inflammatory skin reaction resulting from an outside attack such as a burn, a radiation, a cut, a sting, a graft, or due to an allergen or microbe.

In another preferred embodiment, the disease of the mucosa is selected from the group comprising mucosal psoriasis, candidosis, autoimmune bullous dermatitis, erythema, syphilis, Ducrey's disease, melanoma and disorders such as viral ulcerations and bacterial infections.

In the present invention as depicted herein, the Tr1 cell population is featured by the following specific combination of surface markers: CD4, CD18 and/or CD11a and CD49b. Cd3 can also be contemplated as marker.

Preferably, the Tr1 cell population is a CD3+CD4+CD18brightCD49b+cell population.

The phenotype <<+>> for CD3, CD4, and CD49b molecules means that these molecules or one of its representative fragments are expressed at the surface of said T cells when fluorescent immunoconjugates (such as fluorescent antibodies) are used. The expression <<representative fragment>> means that a fragment of the molecule is present at the surface of the T cells, and that this presence allows to conclude that the molecule is expressed at the surface of the T cells.

The molecule CD18 is characterised to be present at the surface of said Tr1 cells when the intensity of fluorescence obtained for this molecule corresponds to that obtained for the same molecule expressed at the surface of monocytes (<<CD18bright>>).

In a preferred embodiment, the peptide antigen-activated Tr1 cell population is obtained by an in vitro preparation process comprising the following steps:
i) obtaining a Tr1 cell population from the CD4+ T lymphocyte population of the mammal in need of the treatment;
ii) in vitro activating the Tr1 cell population by contacting it with the peptide antigen; and
iii) recovering the peptide antigen-activated Tr1 cell population.

Preferably, the step i) of obtaining the Tr1 cell population comprises the following steps:
a) isolating a progenitor cell population from said mammal;
b) obtaining a population of dendritic cells by culturing said progenitor cell population in presence of interleukine-10 (IL-10);
c) contacting cells of step b) with the CD4+ T lymphocyte population isolated from said mammal to allow differenciation of said CD4+ T lymphocytes into the Tr1 cell population; and
d) recovering the Tr1 cell population from the step c).

In step b), IL-10 is from 50 to 250 Uml⁻¹, preferably at 100 Uml⁻¹ in the culture medium.

The obtention of the Tr1 cell population with steps comprising contacting dendritic cells with a CD4+ T lymphocyte population, and obtaining the population of dendritic cells by culturing said progenitor cell population in presence of interleukine-10 (IL-10), are described in the paragraphs “Results” and “Experimental procedures” of the publication Waickach et al. (Immunity. 2003 May; 18(5):605-17), incorporated herein by reference.

In yet another preferred embodiment, step i) which is obtaining the Tr1 cell population comprises the following steps:
a) contacting the CD4+ T lymphocyte population with an appropriate amount of alpha-interferon (α-IFN); and
b) recovering the Tr1 cell population.

α-IFN is preferably at 5 ng/ml in the media.

In the step a), the media may further comprise an appropriate amount of IL-10, which is preferably at 100 Uml-1.

In step b), the Tr1 cell population may be cultured in a media comprising interleukine 15 (IL-15) to allow proliferation. IL-15 is preferably at 5 ng/ml in the media.

The process of obtaining a Tr1 cell population by contacting a CD4+ T lymphocyte population with an appropriate amount of alpha-interferon (α-IFN) is described in the paragraph “Tr1 cell differentiation” of the american patent application US 2002/0034500, which was published on Mar. 21, 2002 (LEVINGS et al) (see from p. 2, col. 2, L. 33 to p. 6, col. 1, L. 22 and claims, which are incorporated herein by reference).

In still another embodiment, the peptide antigen-activated Tr1 cell population is obtained by an in vitro preparation process comprising the following steps:
i) in vitro activating the CD4+ T lymphocyte population in presence of the peptide antigen, presented by artificial antigen presenting cells; and
ii) recovering an activated CD4+ T lymphocyte population comprising at least 10% of the peptide antigen-activated Tr1 cell population.

Preferably, the artificial antigen presenting cells express a HLA II system molecule and a human LFA-3 molecule, and don't express the co-stimulation molecules B7-1, B7-2, B7-H1, CD40, CD23 and ICAM-1.

The preparation process of obtaining the antigen-activated Tr1 cell population wherein artificial antigen presenting cells are used is described in the international patent application WO 02/092793 published on Nov. 21, 2002, from page 5, L. 8 to 14, L. 25, which passage is incorporated herein by reference. The FIG. 1 of this patent application is also incorporated herein by reference.

In still another embodiment, the peptide antigen-activated Tr1 cell population is obtained by an in vitro preparation process comprising the following steps:
i) in vitro activating the CD4+ T lymphocyte population in presence of the antigen and an appropriate amount of interleukine-10 (IL-10); and
ii) recovering the peptide antigen-activated Tr1 cell population.

Preferably, IL-10 is present in the culture media at a 100 Uml⁻¹.

The preparation process of obtaining the antigen-activated Tr1 cell population wherein IL-10 is used is described in the scientific publication Groux et al (Nature. Oct. 16, 1997; 389(6652):737-42) in the paragraph “Methods”, which is incorporated herein by reference.

In another embodiment, the Tr1 cell population is obtainable by any method using said markers. For example, Tr1 cells can be identified and/or purified by Elisa, flow cytometry, immunoaffinity chromatography with: antibodies directed against said markers, for example with:

APC-conjugated anti-CD4 (RPA-T4)—Becton Dickinson

PC5-conjugated anti-CD3 (UCHT-1)—Caltag

PE-conjugated anti-CD18 (6.7)—Becton Dickinson

FITC-conjugated anti-CD49b (AK-7)—Becton Dickinson

Purification of CD3+CD4+CD18brightCD49b+ Cells:

Enrichment of CD3+CD4+CD18brightCD49b+ cells from a T cell population can be performed with magnetic beads in two steps:

depletion of the total population with anti-human Ig-magnetic beads of cells bound with human anti-CD8, anti-CD14, anti-CD56 and anti-CD19, and

selection of CD49b+ cells bound to an anti-CD49b human antibody with anti-human Ig-magnetic beads.

Further purification is possible with flow cytometry or beads with CD3, CD18 and CD49b antibodies.

ELISA tests may also be used to measure IL-4, IL-10, and IFN-alpha expression.

The Tr1 cell markers and their applications for identifying that population of T cells are widely disclosed in the patent application WO/FR 004/001583, which is incorporated herein by reference (especially claims and example 5).

In the present invention, the mammal in need of such a treatment is preferably a human being.

A second aspect of the present invention is aimed at a pharmaceutical formulation comprising the lipopeptide of the present invention, together with a pharmaceutically topical acceptable carrier, wherein said lipopeptide comprises a peptide antigen specific for a T cell population, said peptide antigen being coupled covalently with a lipid radical and being capable of activating the T cell population.

Preferably, the T cell population is a CD8+ T cell population, a CD4+ T cell population or a Tr1 cell population.

In one embodiment, the pharmaceutical formulation further comprises, as a combined preparation, the peptide antigen or a polypeptide comprising the peptide antigen to be administered prior to the topical administration of the lipopeptide in an immunisation step. Preferably, the prior immunisation is made subcutaneously or intraperitoneally

In another embodiment, the pharmaceutical formulation further comprises, as a T cell population, a Tr1 cell population obtained from a CD4+ T cell population of said mammal, said lipopeptide and said Tr1 cell population being administered simultaneously, separately or sequentially to said mammal.

In a preferred embodiment, the invention is directed to a pharmaceutical composition as defined above, wherein the peptide antigen-activated Tr1 cell population which is administered to the mammal is from 10⁶ to 10⁹ cells/kg. More preferably, the peptide antigen-activated Tr1 cell population which is administered to the mammal is from 0.5 10⁷ to 1.5 10⁷ cells/kg, most preferably 10⁷ cells/kg.

In another embodiment, the invention is directed to a pharmaceutical composition as depicted above comprising:

topical administration of the lipopeptide at the inflammation site, and

intravenous, intramuscular, intra-arterial, intramedullary, intrathecal, intraventricular, transdermal or subcutaneous administration of the peptide antigen-activated Tr1 cell population.

In still another embodiment, the pharmaceutical composition as depicted above is for treating or preventing a mammal suffering from a disease selected from the group of skin diseases and diseases of the mucosa

Preferably, the skin disease is selected from the group comprising psoriasis, vitiligo, prurigo, pityriasis, eruptive cutaneous mastocytosis, scleroderma, bullous dermatitis, cutaneous emphysema, eryhtema, eczema, acne, oedema, graft rejection and melanoma. The skin disease may be also a local inflammatory skin reaction resulting from an outside attack such as a burn, a radiation, a cut, a sting, a graft, or due to an allergen or microbe.

Another preferred embodiment is the pharmaceutical composition as depicted above wherein the disease of the mucosa is selected from the group comprising mucosal psoriasis, candidosis, autoimmune bullous dermatitis, erythema, syphilis, Ducrey's disease, melanoma and disorders such as viral ulcerations and bacterial infections.

Still another preferred embodiment is the pharmaceutical composition as depicted above wherein the Tr1 cell population is a CD3+CD4+CD18brightCD49b+ cell population.

A third aspect of the present invention is aimed at the use of a lipopeptide or a mixture thereof, wherein said lipopeptide comprises a peptide antigen specific for a T cell population, said peptide antigen being coupled covalently with a lipid radical and being capable of activating the T cell population, for the manufacture of a topical medicinal product for treating or preventing a disease selected from the group of skin diseases and diseases of the mucosa

Preferably, the use as depicted above further comprises as a combined preparation, the peptide antigen or a polypeptide comprising the peptide to be administered prior to the topical medicinal product in an immunisation step.

A fourth aspect of the present invention is aimed at a cosmetic formulation comprising a lipopeptide or a mixture thereof, wherein said lipopeptide comprises a peptide antigen specific for a T cell population, said antigen being coupled covalently with a lipid radical and being capable of activating the T cell population, together with a cosmetically acceptable carrier, to prevent or treat disorders selected from chronic inflammatory disorders associated with ageing and its effects, auto-immune pathological disorders,.

Said cosmetic formulation is also advantageously used to delay the accelerated ageing of the skin subject to outside attacks, particularly to prevent photoinduced skin ageing.

The outside environment continuously attacks the skin, whether via ultraviolet radiation or via the radiation emitted by discharge lamps or the various atmospheric natural antigens or those existing due to human activity, urban pollution, etc., which initiates biological natural ageing acceleration processes. In this way, the anti-inflammatory system is continuously active, inducing an acceleration in skin keratinocyte renewal, or even hyperproliferation, aggravating tissue entropy due to an over-expression of specific proteins and, in the long term, a loss of functionality. This results in renewal exhausting the natural keratinocyte reserves and the induction of premature skin ageing. The cosmetic formulation according to the invention advantageously makes it possible to inhibit inflammatory disorders and thus prevent skin ageing. The cosmetic formulation according to the invention advantageously comes in solid, pasty or liquid form.

Cosmetically acceptable carriers are well known by the man skilled in the art, and some of these carriers are the same that those described above as pharmaceutically topical acceptable carriers.

The legends of the figures and examples given below are intended to illustrate the invention, without restricting the scope thereof in any way.

### LEGENDS OF THE FIGURES

FIG. 1: Tr1 cells accelerate the remission of hapten-mediated skin inflammation.

Lipopeptide activates T lymphocytes in vivo. BALB/c mice were injected intravenously with 20×10⁶ DO11-10 TCR-anti OVA transgenic splenocytes. Mice where then treated during 4 days by applying daily 20 μl of 50 μM OVA323-339-lipopeptide or the vehicle directly on one ear. At day five, mice were sacrificed, the draining lymph node and the contra-lateral node cells were stained with the anti-idiotype KJ1-26 recognizing specifically DO11-10 T lymphocytes, anti-CD4 and anti-CD25 antibody. FACS analysis is shown for lymph node cells gated on CD4⁺ T lymphocytes.

FIG. 2:

BALB/c mice were treated with the hapten Oxazolone (1 mg/ear) at day 0, 1 and 2. At day 3, all mice received one million of either Tr1, Th1 or Th2 T cell populations or the Tr1 clone (A-10-9) intraperitoneally. Mice were then treated during 6 days by applying daily 20 μl of 50 μM OVA323-339-lipopeptide ( ) or vehicle (▪) directly on the inflamed ear. Results are shown as the mean ± SD of the thickness of inflamed ears of one representative experiment out of two performed.

FIG. 3: Induction of a CD8⁺ mediated T lymphocyte response in vivo by epicutaneous lipopeptide application.

FIG. 4: Enhancement of a CD8⁺ mediated T lymphocyte response in vivo by epicutaneous lipopeptide application.

FIG. 5: Enhancement of a CD4⁺ mediated humoral response in vivo by epicutaneous lipopeptide application

### EXAMPLES

### Example 1

1.1 Material and Methods

Mice

Specific pathogen-free BALB/c and C.B-17 scid mice were obtained from CERJ (Le Genest Saint Isle, France). Homozygous DO11-10 mice were a generous gift from Dr. S. D. Hurst (DNAX Research Institute, Palo Alto, Calif.). Mice were maintained in our animal facility. C.B-1 7 scid mice were housed in microisolator cages with sterile filtered air (Rec Biozone, Margate, UK). Female mice were used at 8-12 weeks of age.

Antibodies, Media and Reagents

The medium used for T cell cultures was Yssel medium (24) supplemented with 10% FCS (Roche, Meylan, France) and 2×10⁻⁵ M β2 mercaptoethanol (from Invitrogen, San Diego, Calif.). Recombinant mouse IL-10 and ILA were gifts from Dr R. L. Coffman (DNAX Research Institute, Palo Alto, Calif.). Recombinant mouse IFN-γ and IL-12 were purchased from R&D Systems (Minneapolis, Minn.). Purified anti-IL-4 (11B11), anti-IL-10 (2A5), anti-IFN-γ (XGM1.2) and biotin anti-ILA4 (24G2), anti-IL-10 (SXC1), and anti-IFN-γ (R4-6A2; all from BD PharMingen, Le Pont de Claix, France) were used for cytokine assays. The following monoclonal antibodies were used for mouse cell detection and purification: anti-I-A^{d} (AMS-32.1) anti-CD8 (53-6.7), anti-CD11b (M/70), anti-B220 (RA36B2), FITC-conjugated anti-mouse CD45RB (16A), TC- or PE-conjugated anti- CD4 (GK1.5), PE-conjugated anti-CD62L (Me1-14), FITC-conjugated anti-CD25 (7D4), FITC- or biotynylated-KJ-1.26 mAb revealed by PE-labeled steptavidin, FITC- and PE-conjugated isotype control antibodies (BD-PharMingen). The following antibodies were used in vivo, GL113 (isotype control, rat IgG1) and 1B1.2 (anti-mIL-10 R, provided by Dr. K. Moore, DNAX Research Institute). For in vivo use, mAb were purified by column chromatography from tissue culture supernatants. The resulting antibodies were >98% pure and contained <3 endotoxin units of endotoxins per mg of protein. Lysis buffer, OVA ₃₂₃₋₃₃₉ peptide, Ovalbumin, and oxazolone were from Sigma-chemie (Saint Quentin Fallavier, France). OVA₃₂₃₋₃₃₉-lipopeptide was purchased from Bachem (Voisin-le-Bretonneux, France).

T-Cell Populations and T-Cell Clones

The mouse T-cell clones were obtained from DO11-10 mice after in vitro differentiation as previously described (Groux et al., 1997). Naive (MEL-14^{bright}) CD4⁺, KJ-1.26⁺ cells were stimulated for 3 weeks repeatedly with OVA₃₂₃₋₃₃₉ peptide in the presence of IL-4 and anti-IL-12, IL-12 and anti-IL-4 or IL-10 for Th2, Th1 or Tr1 cells respectively. The populations obtained were either used ill vivo or cloned at one cell/well by cytofluorometry (FACS vantage SE, Becton BD Biosciences) and stimulated with irradiated splenocytes (4500 rad) and OVA peptide. Clones were then expanded and analyzed for cytokine secretion after activation with APCs and OVA peptide (Table 1). Selected clones were then expanded by stimulation with irradiated splenocytes and OVA peptide every 2 weeks and further expanded with IL-2 (R&D system, 10 ng/ml). T-cell clones were used at least 10 days after the last stimulation.

TABLE 1
Cytokine profile of the different T cell used

| | | IL-2 | IL-4 | IL-10 | IFN-γ |
|---|---|---|---|---|---|
| Name | Type | (pg/ml) | (pg/ml) | (pg/ml) | (ng/ml) |
| A-10-9 | Tr1 | <40 | <50 | 1874 ± 217 | 65 ± 9 |
| A-10-11 | Tr1 | <40 | <50 | 1595 ± 184 | 42 ± 4 |
| Nice-1 | Tr1 | <40 | <50 | 1936 ± 502 | 38 ± 12 |
| Nice-2 | Tr1 | <40 | <50 | 1273 ± 298 | 51 ± 9 |
| N10-7 | Tr1 | <40 | <50 | 1659 ± 432 | 37 ± 7 |
| N10-11 | Tr1 | <40 | <50 | 1804 ± 394 | 41 ± 5 |
| N10-23 | Tr1 | <40 | <50 | 1493 ± 276 | 13 ± 3 |
| N12-4 | Th1 | 219 ± 42 | <50 | <75 | 73 ± 13 |
| N12-8 | Th1 | 275 ± 31 | <50 | <75 | 97 ± 10 |
| N12-13 | Th1 | 196 ± 54 | <50 | <75 | 84 ± 17 |
| N4-2 | Th2 | <40 | 912 ± 81 | 305 ± 49 | <0.2 |
| N4-9 | Th2 | <40 | 1065 ± 103 | 287 ± 36 | <0.2 |
| N4-12 | Th2 | <40 | 715 ± 59 | 412 ± 67 | <0.2 |
| | Pop. Tr1 | <20 | 112 ± 19 | 12865 ± 1457 | 5 ± 0.1 |
| | Pop. Tr1 | <20 | 86 ± 21 | 14945 ± 1065 | 2.8 ± 0.2 |
| | Pop. Th1 | 513 ± 106 | <40 | <75 | 156 ± 4 |
| | Pop. Th1 | 312 ± 95 | <40 | <75 | 124 ± 15 |
| | Pop. Th2 | <20 | 2321 ± 769 | 6378 ± 834 | <0.2 |
| | Pop. Th2 | <20 | 998 ± 143 | 5241 ± 984 | <0.2 |

T cell clones and T cell populations were generated as described previously. T cells (10⁶ cells/ml) were stimulated with OVA peptide (0.6 μM) and irradiated total splenocytes (2×10⁶ cells/ml). Cytokines were analyzed by ELISA in culture supernatants collected after 48 h of culture. For the different T-cell clones results represent the mean ± SD of 3 representative experiments of stimulation. For the T-cell populations, the results represent the mean ± SD of triplicate measurements of two representative experiments.

Contact Sensitivity to Oxazolone

Contact sensitivity to Oxazolone was performed by applying 20 μl of a 50 mg/ml Oxazolone solution in acetone/olive oil (4:1, vol:vol) epicutaneously on the right ear once a day during three days. The left ear received the vehicle only. Ear thickness was monitored every day. OVA₃₂₃₋₃₃₉-lipopeptide was diluted at 50 μM in olive oil. Mice were treated during 6 days by applying daily 20 μl of 50 μM OVA₃₂₃₋₃₃₉-lipopeptide or olive oil directly on the inflamed ear.

1.2 Tr1 Cells Exert an Anti-Inflammatory Effect on Skin Irritation

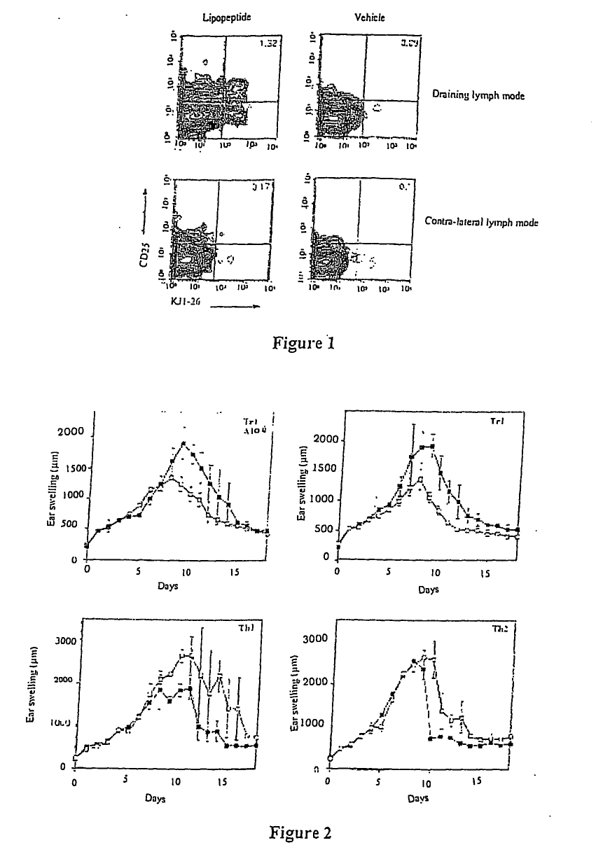

To analyze the potential curative effect of Tr1 cells in a different inflammatory model, the inventors set up skin inflammation experiments using the hapten oxazolone in three daily applications (FIG. 1). Because Tr1 cells need to be activated at the site of inflammation, they first tested the ability of cutaneous application of lipo-Ova peptide to stimulate T cells. BALB/c mice were injected with naive OVA-specific DO11-10 T cells and mice were treated during 6 days by applying daily 20 μl of 50 μM OVA323-339-lipopeptide or olive oil directly on the ear. Analysis of T cells in the draining lymph nodes revealed an accumulation of activated (CD25⁺) OVA-specific (KJ1-26⁺) only in the ear draining-lymph node treated with the lipopeptide (FIG. 1). In that model, three days after the induction of ear inflammation with oxazolone, the mice were treated with OVA-specific Th1, Th2 or Tr1 T-cell populations or a Tr1 cell clone (FIG. 2) and the lipo-OVA peptide was applied for 6 days. In mice treated with Tr1 cells a marked decrease in inflammatory signs was observed whereas treatment with Th1 or Th2 cells enhanced inflammation and oedema (FIG. 2). These results show that the specific regulatory function of Tr1 cells is not restricted to the colon but is also efficient in different tissues and different types of inflammation as previously reported by others (Chen et al., Science. Aug. 26, 1994; 265(5176):1237-40; Barrat et al., J Exp Med. Mar. 4, 2002; 195(5):603-16).

### Example 2

2.1 Material and Methods

Mice

Specific pathogen-free BALB/c and C57BL/6 mice were obtained from CERJ (Le Genest Saint Isle, France). Mice were maintained in laboratory's animal facility. Female mice were used at 8-12 weeks of age.

Antibodies, Media and Reagents

The medium used for T cell cultures was Yssel medium supplemented with 10% FCS (Roche, Meylan, France) and 2×10⁻⁵ M β2 mercaptoethanol (from Invitrogen). Recombinant mouse IFN-γ were purchased from R&D Systems, anti-IFN-γ (XGM1.2), biotin anti-IFN-γ (R4-6A2), anti-IgE (R35-72) (All from Pharmingen Becton Dickinson) were used for cytokine assays. OVA₃₂₃₋₃₃₉ peptide, Ovalbumin, CFA and Alum were from Sigma (Saint Quentin Fallavier, France). TRP2₁₈₀₋₁₈₈ peptide, TRP2₁₈₀₋₁₈₈ lipopeptide, OVA₃₂₃₋₃₃₉-lipopeptide was purchased from Bachem (Voisin-le-Bretonneux, France).

Application of Lipopeptide

TRP2₁₈₀₋₁₈₈ lipopeptide and OVA₃₂₃₋₃₃₉-lipopeptide were diluted at 10 mg/ml in olive oil. Mice were treated during by applying 1 mg lipopeptide or olive oil directly on shaved abdominal skin.

IFNγ and IgE Assays

Sandwich ELISA was used to measure IFN-γ. In brief, ELISA plates (Polylabo, France) were coated with the appropriate coating mAbs (monoclonal antibodies) in carbonate buffer and incubated at 4° C. overnight. Plates were blocked for 30 mn at room temperature with 150 μl of 20% FCS/PBS to each well. 50 μl supernatants from in vitro stimulated cells were then added to the plates and incubated overnight at 4° C. After a washing step, 50 μl/well of the biotinylated second-step Ab was added. Plates were incubated for 1 h at room temperature and washed. The enzyme conjugate (streptavidin-peroxidase) was then added to each well. Plates were incubated at room temperature for 1 h, washed and 100 μl/well of substrate was added, (1 mg/ml ABTS). Plates were read on an ELISA reader at a wavelength of 405 nm after color development (Labsystems iEMS reader, Helsinki, Finland). For OVA specific IgE assays, anti-IgE (R35-72) was used as coating antibody following by sera incubation. Digoxygenine-labeled Ovalbumin was then added to the wells following by Peroxydase-coupled Anti- Digoxygenine

2.2 Activation of a CD8⁺ Mediated T Lymphocyte Response in vivo by Epicutaneous Lipopeptide Application

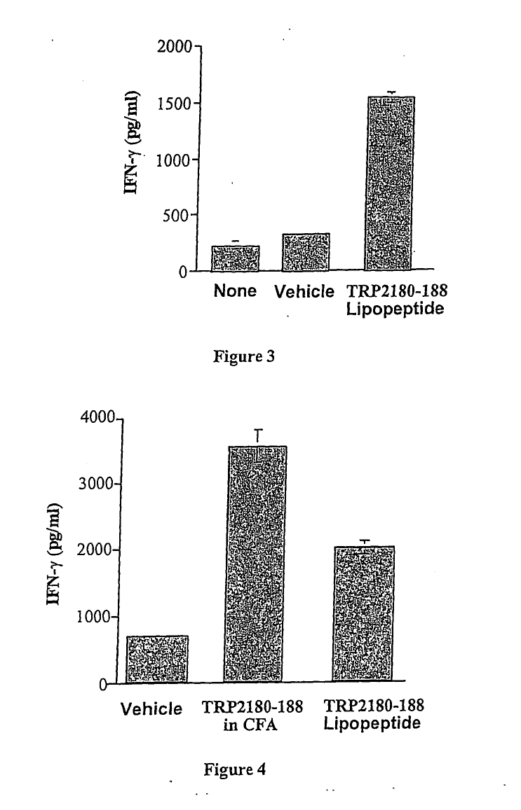

The inventors first wanted to know whether the epicutaneous application of a MHC class I restricted peptide formulated as a lipopeptide could prime a T CD8⁺ lymphocyte mediated immune response in mice. For this purpose they used the lipopeptide TRP2₁₈₀₋₁₈₈ derived from the tyrosinase related protein-2 and restricted to the H2-K^{b} molecule. 1 mg of this lipopeptide was delivered on the skin of the shaved abdomen of C57BL/6 mice once a day during one week. One week after the last lipopeptide application, mice were sacrificed and the production of IFN-γ by skin draining lymph nodes cells was measured in vitro upon restimulation with the TRP2₁₈₀₋₁₈₈ peptide. As shown in FIG. 3, high amounts of IFN-γ were produced by lymph nodes cells in response to the TRP2₁₈₀₋₁₈₈ peptide in lipopeptide treated mice compared with mice untreated or mice treated with the vehicle.

The inventors next wanted to evaluate the capacity of the TRP2₁₈₀₋₁₈₈ lipopeptide treatment to enhance a prior immunisation of mice with the TRP2₁₈₀₋₁₈₈ peptide. Mice were then treated subcutaneously with 25 μg of the the TRP2₁₈₀₋₁₈₈ peptide in complete freund adjuvant (CFA). After one week, one group of mice received subcutaneously 10 μg of the TRP2₁₈₀₋₁₈₈ peptide in CFA, one group was treated 3 days by one application of 1 mg of the TRP2₁₈₀₋₁₈₈ lipopeptide on the shaved abdominal skin and the control group received the vehicle only using the same protocol. At day 30, mice were sacrificed and the production of IFN-γ by skin draining lymph nodes cells was measured in vitro upon restimulation with the TRP2₁₈₀₋₁₈₈ peptide. FIG. 4 show that epicutaneous treatment of mice with the TRP2₁₈₀₋₁₈₈ lipopeptide induce an enhancement of the IFNγ production by lymph nodes cells in previously immunised mice and then is able to enhance a CD8⁺ mediated T lymphocyte response previously induced by TRP2₁₈₀₋₁₈₈ peptide immunisation.

2.3 Enhancement of a CD4⁺ Mediated Humoral Response in vivo by Epicutaneous Lipopeptide Application

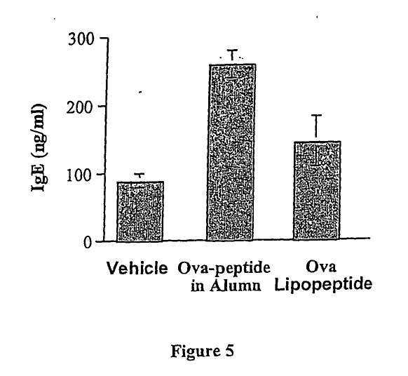

The inventors also wanted to know wether a lipopeptide epicutaneous application could enhance an humoral response elicited by CD4⁺ T lymphocytes in immunised mice. For this purpose they used ovalbumine as the immunising antigen. They also used the peptide 323-339 from Ovalbumin restricted to the MHC Class II-A^{d} molecule for the lipopeptide preparation. Balb/C mice were then immunised intraperitoneally with 25 μg of Ovalbumin in Alumn. After ten days, one group of mice received intraperitoneally 10 μg of Ovalbumin in Alumn, one group was treated 3 days by one application of 1 mg of the Ova lipopeptide on the shaved abdominal skin and the control group received the vehicle using the same protocol. At day 20, mice were sacrificed and the concentration of Ovalbumin-specific IgE immunoglobulin was measured in the serum of mice. As shown in FIG. 5, application of lipopeptide epicutaneously induce an increase in the serum concentration of Ovalbumin specific IgE in mice immunised with ovalbumin.

1. A method of treating or preventing a disease selected from the group of skin diseases and diseases of the mucosa, comprising administering topically to a mammal in need of such a treatment a topical medicinal product comprising a lipopeptide or a mixture thereof, wherein said lipopeptide comprises a peptide antigen specific for a T cell population, said peptide antigen being coupled covalently with a lipid radical and being capable of activating the T cell population.

2. The method of claim 1, wherein the T cell population is a CD8+ T cell population, a CD4+ T cell population or a Tr1 cell population.

3. The method of claim 1, wherein between 1 μg/cm² to 500 ρg/cm², preferably 10 μg/cm², of said lipopeptide is administered topically to said mammal.

4. The method of anyone of claim 1, wherein the lipid radical of the lipopeptide is derived from a fatty acid.

5. The method of claim 4, wherein the fatty acid is palmitic acid.

6. The method of anyone of claim 1, wherein the topical medicinal product further comprises a pharmaceutically topical acceptable carrier.

7. The method of anyone of claim 1, further comprising, prior to the topical administration of the lipopeptide, an immunisation step of the mammal with the peptide antigen or with a polypeptide comprising the peptide antigen.

8. The method of claim 7, wherein the prior immunisation is made subcutaneously or intraperitoneally.

9. The method of claim 1, wherein the peptide antigen has been used to activate in vitro, as a T cell population, a Tr1 cell population obtained from a CD4+ T cell population of said mammal, and wherein said method further comprises the administration of the Tr1 cell population activated by said peptide antigen, the topical administration of the lipopeptide being made sequencially, simultaneously or separately with the administration of the Tr1 cell population.

10. The method of claim 9, wherein the peptide antigen-activated Tr1 cell population which is administered to the mammal is from 10⁶ to 10⁹ cells/kg.

11. The method of claim 10, wherein the peptide antigen-activated Tr1 cell population which is administered to the mammal is from 0.5 10⁷ to 1.5 10⁷ cells/kg, preferably 10⁷ cells/kg.

12. The method of claim 9, comprising intravenous, intramuscular, intra-arterial, intramedullary, intrathecal, intraventricular, transdermal or subcutaneous administration of the peptide antigen-activated Tr1 cell population.

13. The method of anyone claim 1, wherein the skin disease is selected from the group comprising psoriasis, vitiligo, prurigo, pityriasis, eruptive cutaneous mastocytosis, scleroderma, bullous dermatitis, cutaneous emphysema, eryhtema, eczema, acne, oedema, graft rejection and melanoma.

14. The method of claim 13, wherein the skin disease is a local inflammatory skin reaction resulting from an outside attack such as a burn, a radiation, a cut, a sting, a graft, or due to an allergen or microbe.

15. The method of claim 1, wherein the disease of the mucosa is selected from the group comprising mucosal psoriasis, candidosis, autoimmune bullous dermatitis, erythema, syphilis, Ducrey's disease, melanoma and disorders such as viral ulcerations and bacterial infections.

16. The method of claim 1, wherein the topical medicinal product is intended to be administered at the inflammation site.

17. The method of claim 2, wherein the Tr1 cell population is a CD3+CD4+CD18brightCD49b+ cell population.

18. The method of anyone claim 9, wherein the peptide antigen-activated Tr1 cell population is obtained by an in vitro preparation process comprising the following steps:
i) obtaining a Tr1 cell population from the CD4+ T lymphocyte population of the mammal in need of the treatment;
ii) in vitro activating the Tr1 cell population by contacting it with the peptide antigen; and
iii) recovering the peptide antigen-activated Tr1 cell population.

19. The method of claim 18, wherein the step i) of obtaining the Tr1 cell population comprises the following steps:
a) isolating a progenitor cell population from said mammal;
b) obtaining a population of dendritic cells by culturing said progenitor cell population in presence of interleukine-10 (IL-10);
c) contacting cells of step b) with the CD4+ T lymphocyte population isolated from said mammal to allow differenciation of said CD4+ T lymphocytes into the Tr1 cell population; and
d) recovering the Tr1 cell population from the step c).

20. The method of claim 18, wherein the step i) of obtaining the Tr1 cell population comprises the following steps:
a) contacting the CD4+ T lymphocyte population with an appropriate amount of alpha-interferon (α-IFN); and
b) recovering the Tr1 cell population.

21. The method of claim 20, wherein the step a) of contacting is in combination with an appropriate amount of IL-10, such as 100 Um⁻¹.

22. The method of claim 20, wherein the Tr1 cell population is further proliferated in interleukine 15 (IL-15).

23. The method of claim 9, wherein the peptide antigen-activated Tr1 cell population is obtained by an in vitro preparation process comprising the following steps:
i) in vitro activating the CD4+ T lymphocyte population in presence of the peptide antigen, presented by artificial antigen presenting cells; and
ii) recovering an activated CD4+ T lymphocyte population comprising at least 10% of the peptide antigen-activated Tr1 cell population.

24. The method of claim 23, wherein the artificial antigen presenting cells express a HLA II system molecule and a human LFA-3 molecule, and don't express the co-stimulation molecules B7-1, B7-2, B7-H1, CD40, CD23 and ICAM-1.

25. The method of claim 9, wherein the peptide antigen-activated Tr1 cell population is obtained by an in vitro preparation process comprising the following steps:
i) in vitro activating the CD4+ T lymphocyte population in presence of the antigen and an appropriate amount of interleukine-10 (IL-10); and
ii) recovering the peptide antigen-activated Tr1 cell population.

26. The method of claim 17, wherein the Tr1 cell population is purified with Elisa, flow cytometry and/or immunoaffinity using the following antibodies:
anti-CD4, and
anti-CD3, and
anti-CD18, and
anti-CD49b.

27. The method of claim 26, wherein enrichment of CD3+CD4+CD18brightCD49b+ cells from a T cell population comprises the following steps
depletion of the total population with anti-human Ig-magnetic beads of cells bound with human anti-CD8, anti-CD14, anti-CD56 and anti-CD19, and
selection of CD49b+ cells bound to an anti-CD49b human antibody with anti-human Ig-magnetic beads.

28. The method of claim 1, wherein the mammal in need of such a treatment is a human being.

29. A pharmaceutical formulation comprising the lipopeptide of claim 1, together with a pharmaceutically topical acceptable carrier, wherein said lipopeptide comprises a peptide antigen specific for a T cell population, said peptide antigen being coupled covalently with a lipid radical and being capable of activating the T cell population.

30. The pharmaceutical formulation of claim 29, wherein the T cell population is a CD8+ T cell population, a CD4+ T cell population or a Tr1 cell population.

31. The pharmaceutical formulation of claim 29, further comprising, as a combined preparation, the peptide antigen or a polypeptide comprising the peptide antigen to be administered prior to the topical administration of the lipopeptide in an immunisation step.

32. The pharmaceutical formulation of claim 31, wherein the prior immunisation is made subcutaneously or intraperitoneally.

33. The pharmaceutical composition of claim 29, further comprising, as a T cell population, a Tr1 cell population obtained from a CD4+ T cell population of said mammal, said lipopeptide and said Tr1 cell population being administered simultaneously, separately or sequentially to said mammal.

34. The pharmaceutical composition of claim 33, wherein the peptide antigen-activated Tr1 cell population which is administered to the mammal is from 10⁶ to 10⁹ cells/kg.

35. The pharmaceutical composition of claim 33, wherein the peptide antigen-activated Tr1 cell population which is administered to the mammal is from 0.5 10⁷ to 1.5 10⁷ cells/kg, preferably 10⁷ cells/kg.

36. The pharmaceutical composition of claim 33, comprising:
topical administration of the lipopeptide at the inflammation site, and
intravenous, intramuscular, intra-arterial, intramedullary, intrathecal, intraventricular, transdermal or subcutaneous administration of the peptide antigen-activated Tr1 cell population.

37. The pharmaceutical composition of claim 29, for treating or preventing a mammal suffering from a disease selected from the group of skin diseases and diseases of the mucosa.

38. The pharmaceutical composition of claim 37, wherein the skin disease is selected from the group comprising psoriasis, vitiligo, prurigo, pityriasis, eruptive cutaneous mastocytosis, scleroderma, bullous dermatitis, cutaneous emphysema, eryhtema, eczema, acne, oedema, graft rejection and melanoma.

39. The pharmaceutical composition of claim 37, wherein the skin disease is a local inflammatory skin reaction resulting from an outside attack such as a burn, a radiation, a cut, a sting, a graft, or due to an allergen or microbe.

40. The pharmaceutical composition of claim 37, wherein the disease of the mucosa is selected from the group comprising mucosal psoriasis, candidosis, autoimmune bullous dermatitis, erythema, syphilis, Ducrey's disease, melanoma and disorders such as viral ulcerations and bacterial infections.

41. The pharmaceutical composition of claim 30, wherein the Tr1 cell population is a CD3+CD4+CD18brightCD49b+ cell population.

42. A method of manufacturing a topical medicinal product for treating or preventing a disease selected from the group of skin diseases and diseases of the mucosa comprising adding a lipopeptide or a mixture thereof into the topical medicinal product, wherein said lipopeptide comprises a peptide antigen specific for a T cell population, said peptide antigen being coupled covalently with a lipid radical and being capable of activating the T cell population.

43. The method of claim 42, further comprising, packaging as a combined preparation, the peptide antigen or a polypeptide comprising the peptide to be administered prior to the topical medicinal product in an immunisation step.

44. A cosmetic formulation comprising a lipopeptide or a mixture thereof, wherein said lipopeptide comprises a peptide antigen specific for a T cell population, said antigen being coupled covalently with a lipid radical and being capable of activating the T cell population, together with a cosmetically acceptable carrier, to prevent or treat disorders selected from chronic inflammatory disorders associated with ageing and its effects and auto-immune pathological disorders.
